(19) United States
(12) Patent Application Publication
(10) Pub. No.: US 2002/0127593 A1
(43) Pub. Date: Sep. 12, 2002

(54) Fluorescence assay for DNA modifying enzymes

(75) Inventors: Norbert Otto Reich, Santa Barbara, CA (US); Barrett W. Allan, San Diego, CA (US); William Maxwell Lindstrom JR., San Diego, CA (US); Aaron Paul Putzke, Goleta, CA (US)

Correspondence Address: OPPENHEIMER WOLFF & DONNELLY LLP, 38TH FLOOR, 2029 CENTURY PARK EAST, LOS ANGELES, CA, 90067-3024, US

(73) Assignee: Regents of the University of California

(21) Appl. No.: 10/094,364

(22) Filed: Mar. 8, 2002

Related U.S. Application Data
(60) Provisional application No. 60/276,875, filed on Mar. 12, 2001.

(51) Int. Cl. C12Q 1/68; C12P 19/34; C12M 1/34

(52) U.S. Cl. 435/6; 435/91.2; 435/287.2

(57) ABSTRACT

A method of assaying compounds for their ability to effect enzymes including enhancing or inhibiting the effect of those enzymes on double stranded DNA sequences is disclosed. The method comprises providing a modified nucleotide sequence comprised of a base analogue which analogue is characterized by increased fluorescence when moved out of its normal helical position, the sequence having a complimentary sequence hybridized thereto to provide a double stranded sequence. The modified sequence containing the base analogue is brought into contact with the enzyme which enzyme is characterized by effecting the 3-dimensional position of the analogue within the sequence. The enzyme is brought into contact with the sequences in the presence of a compound being assayed. By knowing the amount of increased fluorescence the enzyme would normally have on the sequence is possible to determine the inhibitory or enhancing effect of the compound on the enzyme.

This invention was made, at least in part, with Government support under Grant No. MCB-9603567, awarded by the National Science Foundation. The Government may have certain rights in this invention.

### BACKGROUND OF THE INVENTION

1. Field of the Invention

The field of the invention relates generally to and, more particularly, to a high throughput fluorescent biochemical assay for determining enzyme activity.

2. General Background and State of the Art

The study of protein-DNA complexes reveals diverse mechanisms dependent on sequence-specific interactions between DNA and its binding protein. There are several factors contributing to protein-binding discrimination of DNA, including the DNA base sequence and the geometry of the DNA phosphate backbone (Steitz T A, 1990, Q. Rev. Biophys. 23, 205-280; Pabo P O and Sauer R T, 1992, Annu. Rev. Biochem., 61, 1053-95). For example, many DNA modification and repair enzymes require moving or rotating a base on the DNA molecule in order for proper enzyme-DNA interactions to occur (Klimasauskas P O et al., 1994, Cell, 76, 357-359; Slupphaug G et al.,. 1996, Nature, 384, 87-92). Still, there are enzymes that utilize DNA as a substrate, hence their activity and function is dependent on DNA structure and DNA changes.

Previously, it has been shown that base-specific, DNA conformational changes within protein-DNA complexes can be detected (Allan, B W and Reich, N O, 1996, Biochemistry, 35:147757-14762). Detection of conformational changes is possible by incorporation of 2-Aminopurine (2Ap), a highly fluorescent analogue of adenine, within the DNA duplex (Norlund T M, Andersson S., Nilsson L, Rigler R, Graslund A and McLaughlin L W, 1989, Biochemistry, 28:9095-9103). Similar to adenine, 2Ap forms bonds with thymine (T). Also, 2Ap-substituted DNA molecules retain B-form helical conformation of the DNA.

Studies have shown that enzymes that interact with DNA recognize 2Ap-substituted DNA molecules. For example, EcoRI methyltransferase, an enzyme catalyzing the methylation of adenine-specific nucleotides, is capable of methylating 2Ap-modified DNA molecules similar to other non-modified DNA molecules without 2Ap (Sowers L C, Fazskerley G V, Eritja R, Kaplan B E and Goodman M F, 1986, Proc. Natl., Acad. Sci. U.S.A., 83:5434:5438; Norlund T M, Andersson S., Nilsson L, Rigler R, Graslund A and McLaughlin L W, 1989, Biochemistry, 28:9095-9103; Brennan C A, Van Cleve M D and Gumport R I, 1986, J. Biol. Chem., 261:7270-7278).

Also, it has been shown that 2Ap-substituted DNA molecules are advantageous because they have a greater than 2 fold fluorescence intensity over that of DNA containing only the standard four DNA bases (Allan B and Reich N, 1996, Biochemistry, 35:47, 14757-14762). Moreover, nucleosides and nucleotides incorporating the 2Ap base are highly fluorescent in solution but are strongly quenched upon incorporation into duplex or double-stranded DNA (Allan B, Beechem J M, Lindstrom W and Reich NO, 1998, J. Biol. Chem. 273:2368-73; Ward D C, Reich E and Stryer L, 1969, J. Biol. Chem. 244:1228-37; Bloom L B, Otto M R, Beechem J M and Goodman M F, 1993, Biochemistry 32:11247-58; Allan B, Reich N O and Beechem J M, 1999, Biochemistry 38:5308-5314). Stated another way, 2Ap fluorescence intensity increases dramatically when the 2Ap-substituted DNA molecule denatures (undergoes base-pair opening); or when the 2Ap-substituted DNA molecule goes from a double-stranded molecule to a single-stranded molecule (Jost J P and Saluz H P, 1993, DNA Methylation: Molecular Biology and Biological Significance, Birkhauser Verlag, Basil; Norlund T M, Andersson S., Nilsson L, Rigler R, Graslund A and McLaughlin L W, 1989, Biochemistry, 28:9095-9103).

Further, the 2Ap probe has relatively long excitation λₘₐₓ (˜310 nm) enabling the probe to selectively excite in the presence of protein, therefore, making 2Ap an excellent and widely applicable probe for use in understanding energetics and kinetics of DNA-protein interactions (Allan B, Reich N O and Beechem J M, 1999, Biochemistry 38:5308-5314). For example, because 2Ap fluorescence intensity is highly quenched within double-stranded DNA and EcoRI methyltransferase can methylate 2Ap-modified DNA molecules, 2Ap-modified DNA can be used to study and conformational changes in enzyme-substrate complexes (Allan B, Garcia R, Maegley K, Mort J, Wong D, Lindstrom W, Beechem J M and Reich N O, J. Biol. Chem. 274:19269-75).

Currently, assays that test for enzyme catalytic activity of DNA modifying enzymes are laborious and time consuming. For example, radiometric assays normally used to monitor the incorporation of methyl groups into DNA by DNA methyltransferases typically approach 60 minutes to several hours to process. Radiometric assays are also not cost-effective, and multiple assays can become expensive. The use of radioactivity also leads to problems related to the preparation, storage and disposal of radioactive reagents. The use of radioactivity further leads to an undesirable degree of variability in the performance of the assay as the result of quenching of the radioactivity. Thus a biochemical assay which reduces experimentation time and is more cost-effective is necessary.

Invention Summary

A general object of the present invention is a biochemical assay, to detect and monitor DNA conformational alterations in protein-DNA complexes.

In accordance with one aspect of the present invention, these and other objectives are accomplished by substituting adenine with an adenine fluorescent analogue, including 2-Aminopurine (2Ap).

In accordance with another aspect of the present invention, these objectives are accomplished by providing a biochemical high throughput assay using solution-based fluorescence.

In accordance with another aspect of the present invention, these objectives are accomplished by providing a biochemical high throughput assay using a microtiter approach.

In accordance with another aspect of the present invention, these objectives are accomplished by providing 2Ap to be attached to a matrix including a membrane covering a microtiter plate.

In accordance with another aspect of this invention, these objectives are accomplished by providing a biochemical high throughput assay to monitor and detect DNA modifying enzymes including DNA methyltransferase.

In accordance with another aspect of this invention, these objectives are accomplished by providing a biochemical throughput assay to monitor and detect enzymes other than DNA methyltransferases, including DNA cytosine C5 dimethylases, DNA repair enzymes and other nucleic acid modifying enzymes.

In accordance with another aspect of this invention, these objectives are accomplished by providing a biochemical throughput assay to monitor and detect protein-DNA inhibitors.

In accordance with another aspect of this invention, these objectives are accomplished by providing a biochemical high throughput assay to monitor and detect the activities of enzymes, which utilize DNA as a substrate including DNA polymerases, helicases and endonucleases.

In accordance with another aspect of this invention, these objectives are accomplished by providing incorporation of alternative fluorescent markers.

Lastly, in accordance with an aspect of the present invention, these and other objectives are accomplished by providing a biochemical high throughput assay to monitor and detect candidate small molecules, which alter protein-DNA complexes.

The above described and many other features and attendant advantages of the present invention will become apparent from a consideration of the following detailed description when considered in conjunction with the accompanying drawings.

### DETAILED DESCRIPTION OF THE PREFERRED EMBODIMENTS

While the specification describes particular embodiments of the present invention, those of ordinary skill can devise variations of the present invention without departing from the inventive concept.

This description is not to be taken in a limiting sense, but is made merely for the purpose of illustrating the general principles of the invention. The section titles and overall organization of the present detailed description are for the purpose of convenience only and are not intended to limit the present invention.

One aspect of the present invention is to monitor specific target bases in protein-DNA complexes and to elucidate enzyme function on protein-DNA complexes. There are a number of different types of enzymes known to interact with double-stranded DNA molecules. However, detecting and monitoring DNA modifying enzymes remains difficult. and the precise mechanism of that interaction and its effects vary and are largely unknown because they often are undetected. For example, detection of a single base-flipping occurrence is very difficult under normal circumstances. However, if a base analogue probe containing a high intensity level of fluorescence is used to substitute or replace the target base, then detection is possible.

An ideal fluorescent probe that is capable of being incorporated into DNA is the adenine analogue, 2Ap. 2Ap is ideal because 2Ap base pairs with thymine (T) to form a 2Ap-T base pair. Further, the 2Ap substitution causes minimal structural changes to the DNA molecule (Nordlund et al., 1989, Biochemistry, 28, 9095-9103).

Secondly, in order to study DNA-protein interaction and enzyme function on such interactions, another aspect of the present invention is to use a good candidate protein/enzyme. As stated above, there are a number of enzymes known to interact with double-stranded DNA. To this end, bacterial DNA methytransferases are ideal enzymes for dissecting the molecular basis of sequence-specific DNA changes because they selectively bind to double-stranded DNA and do not bind to single-stranded DNA with any great affinity.

In the present invention, 2Ap-substituted oligonucleotides are constructed to form a double-stranded DNA molecule. Previous studies have shown that double stranded DNA molecules containing 2Ap produce strong fluorescence emission spectrums upon binding with a EcoRI DNA methyltransferase, an enzyme capable of recognizing and displacing the target adenine or 2Ap (Allan B, Garcia R, Maegley K, Mort J, Wong D, Lindstrom W, Beechem J M and Reich N O, J. Biol. Chem. 274:1926975). Further, EcoRI DNA methyltransferases do not bind to single stranded DNA with any detectable affinity, therefore, complementation of top and bottom strand oligonucleotides is required. (Reich N O and Danzitz M, 1992, Biochemistry, 31, 193745). In contrast, adding EcoRI DNA methyltransferase to single stranded DNA containing 2Ap results in only minor (2 fold) increases in fluorescence intensity (Allan B and Reich N O, 1996, Biochemistry, 35:47,14757-14762).

The difference in the level of fluorescence between single-stranded 2Ap-modified DNA versus that of double-stranded 2Ap-modified DNA is explained by a previous report which described 2Ap-T base pairs undergoing spontaneous opening 7-fold more rapidly than standard A-T base pairs (Nordlund et al., 1989, Biochemistry, 28, 9095-9103). That is, double-stranded 2Ap-modified DNA spontaneously denatures to form single-stranded DNA molecules, which are intensely fluorescent. However, because DNA methyltransferases, including EcoRI DNA methyltransferase, do not bind with great affinity to single-stranded DNA, 2Ap must be incorporated into double stranded DNA. This increased fluorescence is attributed to the “base flipping” (2Ap-T base pair) action of the EcoRI methyltransferase on the DNA base, and is consistent with opening of the 2Ap-T base pair as shown previously by Nordlund et al., (Norlund et al.,1989, Biochemistry, 28, 9095-9103).

### EXAMPLE 1

### Assay in General

Double-stranded double stranded nucleotide sequences that are normally acted on by a known enzyme are produced. Substituting the target base for the base analogue (i.e. 2Ap for adenine) modifies the sequence. For example, if EcoRI DNA methyltransferase is used, the adenine analogue, or 2Ap, will be “flipped” by the enzyme. However, the base analogue may be any analogue that increases fluorescence when moved to an extrahelical position.

Those skilled in the art reading this disclosure will recognize that various types of different fluorescent base analogues and fluorescent nucleotide analogues can be used in connection with the present invention. Specific examples are provided here. Others may be found within publications including U.S. Pat. No. 5,763,167 issued Jun. 9, 1998; No. 5,925,517 issued Jul. 20, 1999; No. 5,876,930 issued Mar. 2, 1999; No. 5,723,591 issued Mar. 3, 1998; No. 5,525,711 issued Jun. 11, 1996; and PCT Publication WO 95/31469 issued Nov. 23, 1995 all of which publications are incorporated herein by reference in their entirety.

Other useful analogues include nucleotide analogues such as formycin A, formycin B, oxyformycin B, toyocamycin, sangivamycin, pseudouridine, showdomycin, minimycin, pyrazomycin, 5-amino-formycin A, 5-amino-formycin B, 5-oxo-formycin A, 4amino-pyrazolo pyrimidine, 4,6-diamino-pyrazolo [3, 4d] pyrimidine, 4-amino-6oxo-pyrazolo pyrimidine, 4-oxo-pyrazolo [3, 4d] pyrimidine, 4-oxo-6-aminopyrazolo pyrimidine, 4,6-dioxo-pyrazolo [3, 4d] pyrimidine, pyrazolo [3, 4d] pyrimidine, 6-amino-pyrazolo [3, 4d] pyrimidine, 6-oxo-pyrazolo [3, 4d] pyrimidine. Nucleotide analogues that might be used in the present invention are described in U.S. Pat. No. 5,652,099 issued Jul. 29, 1997 incorporated herein by reference in its entirety.

Double stranded DNA sequences can be comprised of a mixture of natural nucleotides and fluorescent nucleotides, or be comprised completely of fluorescent nucleotides. For example, in some embodiments only one base e.g. adenosine is substituted with an analogue selected from the group consisting of formycin, 2-amino purine, ribonucleoside, and 2,6-diamino ribonucleoside, while the other nucleotides in the sequence are natural. In other embodiments all the purines or alternatively all the pyrimidines are changed from the natural nucleotide to be fluorescent nucleotide analogue. The analogue, which corresponds to the natural target base flipped by the enzyme, is preferably a fluorescent analogue with exception described below.

Double DNA sequences used in the invention may have a sugar-phosphate backbone that is identical to that of a naturally occurring nucleotide sequence. However, in some embodiments it is desirable to provide a modified backbone. Modifying the backbone can result in certain advantages such as nuclease resistance, which can enhance reusability. Various modified backbones are described in European Application EP 0 742 287 A2 published Nov. 13, 1996 which is incorporated herein by reference in its entirety.

### EXAMPLE 2

### High Throughput Assay

The general assay method described above can be carried out even more quickly and efficiently as compared to conventional assays, e.g. those using radioactively labeled methyl groups. Accordingly, the above assay can be referred to as a high throughput assay because performance of the assay can be accomplished, for example, in 96-well microtiter plates or on a microarray. DNA molecules are attached at fixed locations (spots) on microarrays. There may be tens of thousands of spots on an array, each containing a huge number of identical DNA molecules (or fragments of identical molecules), of lengths from twenty to hundreds of nucleotides.

Thus, in some situations it is necessary to assay very large numbers of compounds for which no information is known regarding the ability of any of the compounds to inhibit the activity of an enzyme. In such a situation the following methodology might be employed.

Any large number of compounds could be tested using this method. However, this example refers to 1,000 different compounds.

First, a concentration of any given compound, which would be expected to inhibit a given concentration of a known enzyme, is estimated.

The 1,000 different compounds are then pooled together in an amount such that each will be present at or above the estimated effective concentration.

The pooled composition is then combined with double stranded DNA sequences (as described above) which are targets for the enzymes present in a known concentration.

The same type of determination (described above as regards a single compound) is then made for the pooled composition. More specifically, a determination is made as to whether an increase in fluorescence is observed due to the enzyme flipping fluorescent analogues of the DNA out of position.

If fluorescence increases the same amount regardless of whether the pooled composition is added then it can be concluded that none of the 1,000 compounds in the composition inhibits the enzyme. In such a situation this method has increased the speed and efficiency of the method 1,000 fold. Using the above-described method with only a single compound the procedure would need to be repeated 1,000 times to obtain 1,000 separate negative results.

It may be that one or more of the compounds within the pool of 1,000 or more compounds is capable of inhibiting the enzyme activity. If the enzyme activity is inhibited then there will be no increase in fluorescence when the enzyme and DNA sequences are combined with the pooled composition. These results will tell the researcher that one or more of the compounds present in the pooled composition is an inhibitor. Thus, at this point it becomes important to carry out additional assaying as follows to determine which compound or compounds in the pool provided the positive results.

Alternatively, 500 of the 1,000 compounds are pooled into a first pool. The remaining 500 compounds are pooled into a second pool. The same processing as described above with respect to the 1,000 compounds is then carried out on each of the first pool of 500 compounds and second pool of 500 compounds. It may be that one of the pools of 500 compounds shows no activity, i.e. no ability to inhibit enzyme activity. Thus, that group of compounds can be determined as providing negative results, i.e. inability to inhibit the enzyme. However, the second pool does show that one or more of the compounds of the 500 compounds in the second pool does inhibit the enzyme. When this is known the process is repeated again. More specifically, the 500 compounds making up the second pool of 500 are then used to create two pools with each containing 250 compounds, which are then tested in accordance with the same procedure. The process can be repeated any number of times with pools containing any desired number of compounds. The highest degree of efficiency may be obtained by beginning with any given number of compounds and then pooling all those compounds. When all of the compounds show a negative result no further testing is necessary. However, when the pooled composition shows positive results the high throughput assay is most efficient when the number of compounds in each pool is then divided by two. If the original pool of compounds is particularly rich in terms of providing positive results (i.e. an ability to inhibit the enzymes) then it may be necessary to test a large number of pools. However, it is very likely that at least some of the pooled compositions will provide negative results thereby eliminating the need to test individual compounds within that pool.

Those skilled in the art will recognize that a high throughput assay such as that described here can be carried out using robotics. Specifically, robots can be programmed to extract a given amount of composition from a large number of different containers and pool that amount from each container for testing. When the test results are observed the program directing the robotics can be adjusted accordingly. For example, the pool of 1,000 compounds can become two pools of 500 compounds, four pools of 250 compounds, eight pools of 125 compounds, etc. Those skilled in the art will recognize that this methodology might greatly enhance the efficiency of the present invention.

Other Specific Applications

The analogue 2-Aminopurine (2AP) is a highly fluorescent analog of adenine. Enzymes known as adenine-specific DNA methyltransferases will methylate a natural adenine base. One of these enzymes is EcoRI DNA methyltransferase, which will flip the underlined adenine base or A in the sequence GAATCC. Another enzyme TaqI will flip the underlined A in the sequence TCGA. Further, placement of 2AP adjacent to the target base (i.e.G (2AP)ATTC) can also be used to monitor the base flipping event for the EcoRI methyltransferase enzyme.

Based on the above it will be understood that the sequences used in the assays of the invention need not have a fluorescent analogue at the base which is directly modified. The adjacent base may be moved out of its normal helical position and as such it may be the only base, which needs to be replaced in the sequence to carry out the assay of the invention.

As indicated above the assay of the invention can be used with a number of different enzymes, which flip different bases in different sequences. In another specific example the cytosine-specific DNA methyltransferase M.HhaI could be used. This enzyme modifies the underlined cytosine or C in the sequence GCGC.

The ability of compounds to inhibit the activity of the DNA-repair enzyme, T4 endonuclease could also be assayed using the present invention.

DNA methyltransferases, DNA cytosine C5 demethylases, DNA repair enzymes, and other nucleic acid modifying enzymes that either flip, or are likely to “flip out” their target base (e.g., RNA deaminases) are all amenable to the assay technology described here. Since base flipping is required for catalysis for such enzymes, modulation of this event by any molecule (e.g., activators or inhibitors) will logically alter the enzyme's ability to undertake its normal catalytic cycle. The base-flipping assay can also be used to detect the formation of the protein-DNA complex and inhibitors that alter this process can also be detected with this approach. Molecules identified to alter base flipping will be modulators of the target enzyme's catalytic effectiveness. Enzymes that do not necessarily flip out a target base, but use DNA as a substrate, are also amenable to this approach—for example, DNA helicases, DNA polymerases, and DNA endonucleases.

A solution-based fluorescence assay of the invention can be applied to a microtiter-based approach. Oligonucleotides with the appropriately positioned 2AP can be attached to a matrix (e.g., a membrane covering a microtiter plate) and upon addition of the designated enzyme, the fluorescence increase detected. Alternatively, the assay can be carried out entirely in solution without attachment of the 2AP-containing DNA. Modulation of this fluorescence enhancement by addition of candidate molecules (e.g., members of a combinatorial library) can be detected as an alteration (increase or decrease) in the fluorescence change. This microtiter-based approach can be used with a commercially available fluorimeter.

The particular amount or concentration of the double stranded analog containing sequence can vary over a wide range. Amounts sufficient for detection of the increased fluorescence must be used. The DNA containing the 2AP analog was used at 50-500 nanomolar concentrations.

Extensions of this basic approach could include incorporation of additional fluorescent probes (e.g., rhodamine) to either the 5′ or 3′ ends of the DNA. This approach can be used to monitor both the initial DNA binding event by the protein, as well as the base-flipping event. Also, fluorescence measurements involving anisotropy or lifetime measurements provide additional embodiments to the invention making is possible to determine how candidate small molecules alter more subtle aspects of the protein-DNA complex.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

With respect to the claims, it is applicant's intention that the claims not be interpreted in accordance with the sixth paragraph of 35 U.S.C. §112 unless the term “means” is used followed by a functional statement.

We claim:

1. A high throughput method of assaying compound(s) by determining their ability to modify the activity of an enzyme, comprising:
providing a modified double-stranded oligonucleotide sequence, the sequence having a complimentary sequence hybridized thereto to provide a double stranded sequence, the double-stranded oligonucleotide sequence further comprising an incorporated base analogue characterized by increased fluorescence when moved out of its normal helical position;
contacting the modified double-stranded oligonucleotide sequence with an enzyme, said enzyme characterized by affecting the 3-dimensional position of the base analogue within the modified double-stranded oligonucleotide sequence, whereby the contacting is carried out in the presence of a compound(s) to be assayed; and
determining whether the compound(s) affect(s) the enzyme based on a change in the levels of fluorescence of the incorporated base analogue.

2. The method of claim 1, wherein the double stranded sequence is attached to a surface.

3. The method of claim 2 wherein the surface is a non-porous planar surface.

4. The method of claim 1 wherein the assay is performed using a microarray.

5. The method of claim 1, wherein the assay is performed using robotics.

6. The method of claim 1, wherein the double stranded sequence is in solution.

7. The method of claim 1, wherein the enzyme is a methyltransferase.

8. The method of claim 7, wherein the methyltransferase is a cytosine-specific DNA methyltransferase.

9. The method of claim 8, wherein the cytosine-specific DNA methyltransferase is M.HhaI.

10. The method of claim 1, wherein the enzyme is a DNA-repair enzyme.

11. The method of claim 1, wherein the enzyme is a DNA modifying enzyme.

12. The method of claim 1, wherein the analogue can substitute specifically for a natural base in an enzymatic reaction involving nucleic acid replication, ligation and phosphorylation.

13. The method of claim 1, wherein the analogue is selected from the group consisting of formycin, 2-amino purine, ribonucleoside, and 2, 6-diamino ribonucleoside.

14. The method of claim 1, wherein the analogue is selected from the group consisting of formycin A, formycin B, oxyformycin B, toyocamycin, sangivamycin, pseudouridine, showdomycin, minimycin, pyrazomycin, 5-amino-formycin A, 5-amino-formycin B, 5-oxo-formycin A, 4-amino-pyrazolo [3, 4d] pyrimidine, 4,6-diamino-pyrazolo pyrimidine, 4-amino-6-oxo-pyrazolo [3, 4d] pyrimidine, 4-oxo-pyrazolo [3, 4d] pyrimidine, 4-oxo-6-amino-pyrazolo [3, 4d] pyrimidine, 4,6-dioxo-pyrazolo [3, 4d] pyrimidine, pyrazolo [3, 4d] pyrimidine, 6-amino-pyrazolo [3, 4d] pyrimidine, and 6-oxo-pyrazolo pyrimidine.

15. A method for determining the amount of a compound required to inhibit an enzyme that interacts with DNA comprising the steps of:
(a) combining a substrate with a liquid sample containing a known amount of an enzyme that interacts with DNA and a compound being assayed wherein the substrate has attached to a surface a known number of double stranded sequences which sequences comprise a base analogue characterized by increased fluorescence when moved out of its normal helical position;
(b) allowing the enzyme, compound and sequences to interact over a sufficient period of time and under conditions such that the enzymes would move the analogue out of its normal helical position;
(c) determining an increase in fluorescence relative to the known amount of fluorescence of the sequences;
(d) repeating steps (a), (b) and (c) with different amounts of the compound until the amount of the compound needed to prevent an increase in fluorescence is determined by determining the amount of the compound required to inhibit the enzyme.

16. The method of claim 15 wherein the assay is performed using a microarray.

17. A method of assaying a plurality of compounds by determining their ability to modify the activity of an enzyme comprising:
(a) providing a modified double-stranded oligonucleotide sequence, the sequence, the sequence having a complimentary sequence hybridized thereto to provide a double stranded sequence, the double-stranded oligonucleotide sequence further comprising an incorporated base analogue characterized by increased fluorescence when moved out of its normal helical position;
(b) contacting the modified double-stranded oligonucleotide sequence with an enzyme, said enzyme characterized by affecting the 3-dimensional position of the base analogue within the modified double-stranded oligonucleotide sequence, whereby the contacting is carried out in the presence of a plurality of the compounds;
(c) determining whether any of the compounds in the plurality of compounds affects the enzyme based on a change in the levels of fluorescence of the incorporated base analogue.
(d) if there is no change concluding that none of the compounds modifies the activity of the enzyme;
(e) if there is a change dividing the compounds into two or more pools and assaying each pool separately by steps (a)-(c);
(f) eliminating inactive compounds based on the results of the assays of the pools of step (e);
(g) further dividing the remaining compounds into smaller pools and repeating the assay of steps (a)-(c) until the identity of the compounds having the ability to modify the activity of the enzyme is known.

18. The method of claim 17 wherein the plurality of compounds includes at least 100 compounds.

19. The method of claim 18 wherein the plurality of compounds includes at least 1000 compounds.

20 The method of claim 17 wherein the assay is performed using a microarray.
